(19) United States
(12) Patent Application Publication
(10) Pub. No.: US 2009/0082616 A1
(43) Pub. Date: Mar. 26, 2009

(54) DEVICE AND METHOD FOR PROVIDING A MEDITATION EXPERIENCE

(76) Inventor: Zachary Jay Selig, West Hollywood, CA (US)

Correspondence Address: ITALIA IP, 3500 WEST OLIVE AVE., SUITE 300, BURBANK, CA, 91505, US

(21) Appl. No.: 11/858,866

(22) Filed: Sep. 20, 2007

(51) Int. Cl. A61M 21/02

(52) U.S. Cl. 600/27

(57) ABSTRACT

A device for providing a meditative experience wherein the device utilizes an animated production that is generally 10 minutes in length and that focuses on the activation of the seven Chakras as a means of providing physical and/or spiritual wellness. The benefits of the device are automatic such that no prior training is required by the user to realize the wellness effect of the device. The user simply needs to view an animated production where the seven rays activate the seven chakras in a specific manner. The production can be viewed on any form of personal display device and can take place anywhere it is desirable to have a meditative experience.

### BACKGROUND OF INVENTION

This application relates generally to meditation devices. More specifically, this application relates to a device that allows a user to achieve a meditative experience in a considerably shorter amount of time than with conventional techniques and without the level of training and instruction as is generally required.

### SUMMARY

Emotional, psychological stress caused by the increasing pace and demands of modern life is rapidly becoming a serious medical problem. Many somatic ailments, such as high blood pressure, strokes and heart attacks, are rooted in such stress, and other problems, such as migraine headaches, are sometimes aggravated or wholly caused by it.

Various techniques for relieving stress involve some form of relaxation or meditative state, often with the intention of creating a physical and emotional state corresponding to that of a trance. Ways to create this meditative state can range from special sensory-deprivation chambers in which the patient or subject is wholly without external stimulus to other methods where various cues are utilized to create the meditative state. Described herein is a meditation device and method that is purely sensory and non-intellectual in application. The device provides the same level of relaxation in just a few minutes when compared to traditional meditative techniques which can take 40 minutes or more. More specifically, the device utilizes an animated production that is generally about 10 minutes in length and that focuses on the activation of the seven chakras as a means of providing physical and/or spiritual wellness. The benefits of the device are automatic such that no prior training is required by the user to realize the wellness effects of the device. The user simply needs to view an animated production where the seven rays activate the seven chakras in a specific manner. The production can be viewed on any form of personal display device and can take place anywhere it is desirable to have a meditative experience.

In particular, this application discloses a device for providing a meditative experience, the device comprising: an animated production that utilizes the seven rays to activate the seven chakras, wherein the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra; means to view the animated production on a personal display device; and, means to transmit the animated production to a personal display device.

This application also discloses an automatically deliverable meditation method, the method comprising: providing an animated production to a user that utilizes the seven rays to activate the seven chakras, wherein the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra; transmitting the animated production to a personal display device; and, viewing the animated production on personal display device.

This application further discloses a device for providing an automatic meditative experience for a user, the device comprising: an animated production that utilizes the seven rays to sequentially activate the seven chakras, wherein the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra; wherein the production includes visual effects whereby a user is led to follow a moving object through a spatial environment such that a feeling of movement is created in the user by watching it and whereby the production includes successive steps such as to create a beginning and an end; means to view the animated production on a personal display device; and, means to transmit the animated production to a personal display device.

### DETAILED DESCRIPTION

Provided below are four examples of an animated production that can be utilized to provide a meditative experience, the device comprising: an animated production that utilizes the seven rays to activate the seven chakras, wherein the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra. The rays may activate the chakras in any order, however, preferably the rays activate the chakras sequentially according to the following order:
A. The Orange Ray into The Spleen Chakra
B. The Yellow Ray into The Navel Chakra
C. The Green Ray into The Heart Chakra
D. The Blue Ray into The Throat Chakra
E. The Indigo Ray into The Brow Chakra
F. The Violet Ray into The Crown Chakra

The animated production may be produced using traditional known animation techniques or later discovered techniques. Computer generated imagery (CGI) that is 3D is particularly effective in creating visual effects whereby a user is led to follow a moving object through a spatial environment such that a feeling of movement is created in the user by watching it and whereby the production includes successive steps such as to create a beginning and an end. As discussed more fully below in the Examples provided, a user is able to take a wellness journey by simply viewing the animated production as described. To that end, the device includes means to view the animated production on a personal display device and means to transmit the animated production to a personal display device.

As used herein, the term “personal display device” includes any device that a user can employ that has both audio and visual capabilities, such items include, but are not limited to televisions, computers, phones, personal digital assistants, iPods (and similar devices), and devices not personally owned by the user such as audio/visual displays in airplanes, kiosks, and other public locations.

Means to transmit the animated production to a personal display device include storing the animated production on a digital storage device known or later discovered that is playable on the personal display device. Such digital storage devices include, but are not limited to DVDs, CDs, flash drives, VCR tapes and any other such devices that are capable of storing an animated production such that it can be played or transmitted to a personal display device. Additional means to transmit the animated production to a personal display device include any known or later discovered method but may include hosting the production on a user accessible website as is common in the art, wireless transmission, satellite, cable, antennae and any other such means that are capable transmitting the animated production to a personal display device.

### EXAMPLE I

### The Violet Flame Cleanse—Cleanses and Protects the Physical and Spirit Bodies as the Essential Preperation for Kundalini Activation

Time: 10 Minutes

A solid black screen first appears. There is silence.

Music begins with a red star pulsing on the left stage and then a white star pulsing on the right stage. The two stars radiate light, PULSE, and grow larger while gradually moving in an evocative slow dance following one another clockwise, building faster into a heated frenzy like the integral structure of an atom, and then unite into an explosion of white light with a golden aura that dissolves the entire screen white.

Dissolve from complete white into The Great Central Sun.

The Great Central Sun appears right stage and then dissolves into The Blue Ray Painting right stage in a dark blue space of sky with stars and galaxies, which is positioned at a 45-degree angle in front and over the Mind/Spirit/Body Chart left stage that is in a world anchored to the earth, which is blue-green with a partial cloud-cover like it truly looks from space with a dark blue sky filled with shimmering diamond like stars and galaxies.

The lower Physical Body in this chart is built in an anatomical layering of bones, organs, arteries, blood vessels, and with translucent flesh in a Da Vinci-esque humanoid manner that is much more elaborate than the existing rendering. Preferably the Spinal Column should be strongest representation as all Chakras are built upon it. The Blue Ray undulates, PULSES, and spins life-like clockwise and radiates Blue Bubbles that is symbolic of rays throughout the screen. A laser-beam of blue light in the form of Blue Bubbles is projected from the center of The Blue Ray Painting right stage as the camera pans to left stage in a diagonally into following the Blue Bubble flow into an illuminated fully animated Throat Chakra at the throat of the Physical Body represented in the Physical Body of the Mind/Spirit/Body Chart.

1. The Blue Bubbles of laser-beam spirals in a spiraled pink and dark blue tunnel of virtual reality that is built into the animation of the design elements from The Throat Chakra and the Blue Bubbles hits and pierces a closed lotus bud that opens to a full bloom lotus of The Throat Chakra, where The Throat Chakra PULSES brighter and appears activated with slow clockwise spins and undulations of its petals the Blue Bubbles of energy pierce the center of the Sanskrit letter symbol in the middle of the image. The Chakra PULSES and radiates with blue light coming out of the tip of the petals into an aura of blue and gold that surrounds the Chakra on a dark blue background.

The screen opens to a shot of the Blue Bubble energy cascading like a water fall out of a black hole into a grey World of Ether that travels right stage to left stage diagonally.

2. The following frame is that of the Blue Bubble laser energy continuing in a serpentine tunnel descending in a clockwise manner that hits a closed lotus bud, which opens to the animated Heart Chakra.

The Blue Bubbles of energy pierce the center of the Sanskrit letter symbol in the middle of the Chakra image. The Chakra PULSES and radiates with blue light corning out of the tip of the petals into an aura of blue and gold that surrounds the Chakra on a dark blue background.

The screen then opens to a shot of the Blue Bubble energy traveling through a World of Air right stage to left stage diagonally.

3. The following frame is that of the Blue Bubble laser energy continuing in a serpentine tunnel descending in a clockwise manner that hits a closed ‘lotus bud’, which opens to the animated Navel Chakra.

The Blue Bubbles of energy pierce the center of the Sanskrit letter symbol in the middle of the Chakra image. The Chakra PULSES and radiates with blue light corning out of the tip of the petals into an aura of light blue and gold that surrounds the Chakra on a dark blue background.

The screen opens to a shot of the Blue Bubble energy traveling through a World of Fire right stage to left stage diagonally.

4. The following frame is that of the Blue Bubble laser energy continuing in a serpentine tunnel descending clockwise manner that hits a closed lotus bud, which opens to the animated Spleen Chakra. The Blue Bubbles of laser energy pierces the center of the Sanskrit letter symbol in the middle of the image. The Chakra PULSES and radiates with blue light coming out of the tip of the petals into an aura of lighter blue that surrounds the Chakra on a dark blue background. The screen opens to a shot of the Blue Bubble laser energy traveling through a World of Water.

5. The following frame is that of the Blue Bubble laser energy continuing in a serpentine tunnel descending in a clockwise manner that hits a closed ‘lotus bud’, which opens to the animated Root Chakra.

The Blue Bubbles of energy pierce the center of the Sanskrit letter symbol in the middle of the Chakra image. The Chakra PULSES and radiates with blue light coming out of the tip of the petals into an aura of light blue and that surrounds the Chakra on a dark blue background.

There is a dissolve from the auras of The Root Chakra frame into a new frame of a point in the center of The Root Chakra image in the middle of the Sanskrit letter here.

There is an explosion at the middle of The Root Chakra into a fire ball that has a white core with a golden yellow, blue, red, and orange aura.

The Violet Flame appears in the center of this explosion in the center of The Root Chakra.

There is a dissolve from the frame of the explosion into a new frame to show the entire lower Physical Body and the Root Chakra location exploding.

Dissolve back to The Violet Flame inside The Root Chakra center. The Violet Flame expands out over the entire Root Chakra—dissolve back to showing entire Physical Body on the screen with The Violet Flame moving down through the center of the legs and through the soles of the feet of the Physical Body anchoring into the core of the earth. The Violet Flame of energy reverses and moves up the legs to the Root Chakra.

All blue light dissolves as the violet color appears in The Root Chakra.

Note: A cacophony of design elements built from each Chakra that looks like a Kaleidoscope precedes the Chakra image as The Violet Flame pierces each Chakra image.

The Violet Flame then moves up from The Root Chakra (design elements of Spleen Chakra appear in kaleidoscope manner first as the now Purple Bubbles pierce The Spleen Chakra, and expands out in a flame like manner of violet light over the entire Chakra. The process continues up through the remaining 5 Chakras, until all 7 are consumed in The Violet Flame.

As each Chakra is hit The Violet Flame intensifies.

When The Violet Flame has reached a center point at the top of the Physical Body's head it continues to ascend upwards approximately 60 feet through the feet of the Spirit Body of The Mind/Spirit/Body Chart. Ideally, both figures should be shown united as in The Mind/Spirit/Body Chart should be shown in the frame. The light intensifies while moving up the core of golden-white light that connects the Spirit and Physical Bodies in the center of The Violet Flame.

The Violet Flame moves upward through each of the seven Chakras that must be shown in full animation, the same as it has previously done in the Physical Body, until it reaches the top of the center of the head in the Spirit Body.

The camera moves from the lower Physical Body to the upper Spirit Body to identify the two different bodies. The golden-white light moves down and stops at The Heart Chakra of Spirit Body and radiates out of the center of the Heart Chakra beyond the Spirit Body to fill the screen.

At this point at the Heart Chakra the entire Violet Flame expands through and out of the Spirit Body and PULSES/radiates and then the frame shows both upper Spirit Body and lower Physical Body figures anchored into the earth with The Violet Flame.

The Spirit Body of The Mind/Spirit/Body Chart is shown completely radiating brilliant golden-white light from the head, hands, and feet, and all 7 Chakras center points are pulsing in the Spirit Body in the upper figure.

The golden white light from the hands of the Spirit Body shoots down over the Physical Body, creating a tube of white light that goes into the earth around the Physical Body. The Spirit Body of the upper figure in the chart expands and then moves down into the center point of the head of the Physical Body lower figure and explodes the screen in white light at the center of the Heart Chakra point.

The Spirit Body dissolves down into the head of the Physical Body lower figure with The Violet Flame encapsulating both figures.

The Spirit Body now residing inside the Physical Body. There is a slight demarcation in both color and line that shows that there are two bodies within each other first before it dissolves to one body. The Spirit Body PULSES with strong golden white light with a golden aura and then into an explosion of deep gold and then into white light as the Spirit Body unites at the point of The Heart Chakra dissolves into the Physical Body. The golden white light fills the screen and dissolves completely into a white screen.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

### EXAMPLE II

### Solar Kundalini Activation in the Physical Body

Time: 10 Minutes

Start with the above Example I, which leads into the following:

A. Activation of the Kundalini and the Ascent of the Ida and Pingala Nadis—Clearing Knots for the Kundalini Ascent in B.

Opening shot of The Great Central Sun appears stage right on a sky blue screen.

The Great Central Sun dissolves into The Yellow Ray that emits a golden yellow blazing laser-beam of Fahat in the form of bubble energy shooting at a 45 degree angle down directly towards the Navel point of The Physical Body in The Mind/Spirit/Body Chart.

Camera pans with the yellow ray bubbles right stage to left stage to The Mind/Body/Spirit Chart anchored in a daytime world of cerulean blue sky, positioned over a blue green curve of the Earth left stage.

The Yellow Ray of Fahat enters the lower Physical Body at the navel point with an explosion of Golden Yellow.

Dissolve into a closed lotus bud that opens to The Navel Chakra of the Physical Body that shows first a frame of the complete Mind/Spirit/Body Chart format left stage.

Frame dissolves into the yellow bubble rays moving through a World of Fire to ignite the internal Prana or Life Force of Vital Energy of the Cosmos that is Inspirational in the Navel Chakra. Dissolve back to The Navel Chakra.

As the golden yellow laser-beam of bubbles fills The Navel Chakra, the current of light (built as a spiral of bubbles) spirals slightly in counter-clockwise manner down to a point 1 inch below the navel in the perineum, where the Prana with the Apana or Life Force of Vital Wind that is Expirational are mixed. Prana is a dark golden yellow color and Apana is a dark blue color.

The two colored energies ascend with Prana leading Apana in a clockwise. spiraling manner 1 inch up and out to pierce the center of The Navel Chakra where the two mixtures are seeded into a combustion that occurs in an explosion of a green golden white fire.

The green golden fire with a golden center dissolves into a green laser bubble energy that moves clockwise up and out into the closed “lotus bud” that opens to the center of The Heart Chakra in the middle of the Sanskrit letter, where it increases in a green golden pulsing light with waves moving out, into a yellow, orange, and red fire surrounding the green explosion that is fanned by air.

Dissolve to World of Air.

The green light of bubble energy then spirals counter clockwise down through the region of The Navel Chakra where it dissolves back into a Yellow explosion. and then out into the closed lotus bud that opens to The Sexual Chakra that then moves through a World of Water, and down counter clockwise and out into the closed ‘lotus bud’ that opens to center of The Root Chakra that then moves through a World of Earth, where it explodes into a white, yellow, orange and red fire, expanding the 3½ turned coiled Kundalini that now appears as an orange and red fire energy of bubbles expanding The Root Chakra with the base and center of The Root Chakra ignited first with the Ida—white and then the Pingala—red nadis activated.

Dissolve to The Physical Body showing The Root Chakra with the Kundalini expanding and ignited the Ida and the Pingala channels.

The Ida nadi, spirals up first clockwise (white line that begins at the center of the base of The Root Chakra on the left side); and the Pingala nadi, spirals up at the same time, counter clockwise (red line that begins at the center of the base of The Root Chakra on the right side). The two Nadis criss-cross and entwine upward in a spiraling Helix up through the nodes (middle point between and at the base of each Chakra) to a point in the center of The Brow Chakra. An explosion of Blue White Fire occurs at this point of their union in The Brow Chakra, which will precede the Orange and Red Fire of the Kundalini itself, and the Orange and Red Fire go up from The Brow Chakra into The Crown Chakra.

Dissolve to the Physical Body.

The entire Physical Body expands and PULSES with the blue fire light.

B. Ascent of the Kundalini

The Kundalini uncoils slowing and is now thrust upward through the mouth of the central spinal cord in a clockwise spiraling manner piercing (always in and out of spinal column) all closed lotus buds that open the six Chakras, arriving at the top of The Brow Chakra, and then returning its descent counter clockwise down in a reverse order through the Physical Body, piercing the entire lower open Chakra center points back to the base of the spine at The Root Chakra (all Chakra points spin, turning clockwise and counter clockwise, PULSES, undulate, and emit their own color rays with fiery auras as the orange and red fire of bubble energy light hits them—show several revolutions of Kundalini circuitry)

Frame of The Physical Body with The Root Chakra pulsing strongly.

Dissolve back to the Physical Body.

The orange and red current of light of Kundalini then reverses; and, spirals up through each Chakra center point and into The Brow Chakra where it explodes in orange and red fire that pushes an explosion of violet-blue up from its center above through The Crown Chakra and out through the top of the head of the Physical Body. Each Chakra turns into white light with a golden aura when it exits the head. As the laser-beam penetrates each Chakra point in the beginning of the sequence starting in the Heart Chakra, the Chakra petals are closed lotus buds that open to spin clockwise, PULSE, petals undulate, enlarge, and emit rays of light from the petal tips. Each center beginning as a lotus bud and developing into open flowering Chakras with their specific color coding. When the process is completed; the Kundalini fire of orange-red light dissolves into white light with a golden aura as it exits the top of the upper figure. The light then cascades around both the Spirit Body and the Physical Body in a showering of white light cascade down into the earth.

Dissolve back to fiery Kundalini Helix and a descent counter clockwise from The Crown Chakra at the top of the head camera focus of the Kundalini moving to rest and recoils around the center of The Root Chakra as in original 3½ turned position of art format in The Root Chakra.

### EXAMPLE III

### Solar Ray Regeneration in the Physical Body

Time: 10 Minutes

Duplicate the same Kundalini Activation as in Examples I and II.

The Great Central Sun appears at a 45-degree angle center state left, which dissolves into The Red Ray stage right in a world of a daytime blue sky positioned left stage over a blue-green curve of the Earth left stage. The Red Ray emits a laser-beam of ruby red light into The Root Chakra in the Spirit Body of The Mind/Spirit/Body Chart stage left. A laser-beam of red light then moves out of The Root Chakra spiraling in a clockwise direction, creating a pulsing ring of red light with a golden center around The Root Chakra. The process takes place with the following sequential applications:

G. The Orange Ray into The Spleen Chakra

H. The Yellow Ray into The Navel Chakra

I. The Green Ray into The Heart Chakra

J. The Blue Ray into The Throat Chakra

K. The Indigo Ray into The Brow Chakra

L. The Violet Ray into The Crown Chakra

There are 7 ray infusions applied to the 7 Chakras with 7 concentric colored rings surrounding each body as they are infused with red, orange, yellow, green, blue, indigo, and violet.

The entire Physical Body of The Mind/Spirit/Body Chart (center-stage) absorbs all seven colored bands of light into a uniform blazing white light with all seven Chakras pulsating their original color format and projecting white laser-beams of the coded color out. The Physical Body now has beams of white light shooting from the hands, through the top of the Physical Body's head, and with beams of light shooting out of the tips of the fingers that create a pyramid of white light around it and into the earth. The core of the upper figure moves down through the head of the lower figure and rests in the Heart of the physical body and explodes the screen in white light dissolve.

### EXAMPLE IV

### Galactic Ray Regeneration Through the Spirit Body

Time: 10 Minutes

Duplicate the same Kundalini Activation and Solar Regeneration as in Examples I, II, and III that was done in the lower Physical Body to now apply to the upper Spirit Body.

The Great Central Sun appears at a 45-degree angle stage right on a screen of a world of dark blue sky filled with white and gold galaxies and stars pulsing. The Great Central Sun dissolves into The Red Ray stage right. The Red Ray emits a laser-beam of ruby red light into the center of The Root Chakra in the Spirit Body of The Mind/Spirit/Body Chart stage left. A laser-beam of red light then moves out of The Root Chakra spiraling in a clockwise direction, creating a pulsing ring of red light around The Root Chakra. The process takes place with the following sequential applications that will create bands of 7 colors in a rainbow ovoid shape around entire Spirit Body

A. The Orange Ray into The Spleen Chakra

B. The Yellow Ray into The Navel Chakra

C. The Green Ray into The Heart Chakra

D. The Blue Ray into The Throat Chakra

E. The Indigo Ray into The Brow Chakra

F. The Violet Ray into The Crown Chakra

There are 7 Ray infusions into the 7 Chakras.

The entire Spirit Body of The Mind/Spirit/Body Chart (center-stage) moves up higher absorbing all the seven colored bands of light dissolving into a uniform blazing white light with all seven Chakras pulsating their original color format and projecting white laser-beams of the coded color out. The Spirit Body now has beams of white light shooting from the hands, through the top of the upper figure's head, and with beams of light shooting out of the tips of the fingers that create a pyramid of white light around the Physical Body. The core of the Spirit Body moves down through the center of the head of the Physical Body and rests in the Heart of the Physical Body with an explosion of golden white light that fills the screen in a completely golden white light dissolve.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

1. A device for providing a meditative experience, the device comprising:
an animated production that utilizes the seven rays to activate the seven chakras sequentially in the following order: the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra;
wherein the production further comprises visual effects whereby a user is led to follow a moving object through a spatial environment such that a feeling of movement is created in the user by watching it and whereby the production includes successive steps such as to create a beginning and an end;
means to view the animated production on a personal display device; and,
means to transmit the animated production to a personal display device.

2. The device of claim 1 wherein the production further comprises visual effects whereby a user is led to follow a moving object through a spatial environment such that a feeling of movement is created in the user by watching it and whereby the production includes successive steps such as to create a beginning and an end.

3. The device of claim 1 wherein the animated production has a physical effect on the user.

4. The device of claim 1 wherein the animated production has a spiritual effect on the user.

5. The device of claim 1 wherein the animated production has a physical and spiritual effect on the user.

6. The device of claim 1 wherein the activation of the chakras is sequential.

7. The device of claim 1 wherein the means to transmit the animated production is on a digital storage device.

8. The device of claim 1 wherein the means to transmit the animated production is hosting it on a user accessible website.

9. An automatically deliverable meditation method, the method comprising:
providing an animated production that utilizes the seven rays to activate the seven chakras sequentially in the following order: the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra;
wherein the production further comprises visual effects whereby a user is led to follow a moving object through a spatial environment such that a feeling of movement is created in the user by watching it and whereby the production includes successive steps such as to create a beginning and an end;
transmitting the animated production to a personal display device; and,
viewing the animated production on personal display device.

10. The method of claim 1 wherein the production further comprises visual effects whereby a user is led to follow a moving object through a spatial environment such that a feeling of movement is created in the user by watching it and whereby the production includes successive steps such as to create a beginning and an end.

11. The method of claim 9 wherein viewing the animated production has a physical effect on the user.

12. The method of claim 9 wherein viewing the animated production has a spiritual effect on the user.

13. The method of claim 9 wherein viewing the animated production has a physical and spiritual effect on the user.

14. The method of claim 9 wherein the activation of the chakras is sequential.

15. The method of claim 9 wherein step of transmitting the animated production is done by using a digital storage device.

16. The method of claim 9 wherein step of transmitting the animated production is done by using a user accessible website.

17. A device for providing an automatic meditative experience to a user, the device comprising:
an animated production that utilizes the seven rays to activate the seven chakras sequentially in the following order: the red ray activates the root chakra, the orange ray activates the spleen chakra, the yellow ray activates the navel chakra, the green ray activates the heart, the blue ray activates the throat chakra, the indigo ray activates the brow chakra, and the violet ray activates the crown chakra;
wherein the production includes visual effects whereby a user is led to follow a moving object through a spatial environment such that a feeling of movement is created in the user by watching it and whereby the production includes successive steps such as to create a beginning and an end;
means to view the animated production on a personal display device; and,
means to transmit the animated production to a personal display device.

18. The method of claim 17 wherein viewing the animated production also has a spiritual effect on the user.

19. The method of claim 17 wherein step of transmitting the animated production is done by using a digital storage device.

20. The method of claim 17 wherein step of transmitting the animated production is done by using a user accessible website.
